(19) United States
(12) Patent Application Publication
(10) Pub. No.: US 2025/0302600 A1
(43) Pub. Date: Oct. 2, 2025

(54) SYSTEMS AND METHODS FOR THE DELIVERY OF ELECTRICAL CURRENT TO AN ORAL CAVITY

(71) Applicant: JODI VENTURES, INC., KIRKLAND, WA (US)

(72) Inventors: ISSAM NEMEH, WESTLAKE, OH (US); WADI NEMEH, WESTLAKE, OH (US); FADIA NEMEH, WESTLAKE, OH (US); JOSEPH COURY, KIRKLAND, WA (US); DAVID MANDEL, ALEXANDRIA, VA (US); PAUL GANCITANO, WASHINGTON, DC (US); JOSHUA CHIN, ALEXANDRIA, VA (US); CHRISTOPHER TERELLA, STOW, MA (US)

(21) Appl. No.: 18/621,907

(22) Filed: Mar. 29, 2024

(51) Int. Cl. A61D 5/00; A61N 1/05; H01B 17/56

(52) U.S. Cl. CPC A61D 5/00; H01B 17/56; A61N 1/0548

(57) ABSTRACT

Systems and methods according to the present invention include a device for delivering non-invasive direct current electricity to the oral cavity of a non-human animal. The device generally comprises layers, wherein an electronic circuit assembly is protected within an enclosure, which itself is protected within a frame. Surrounding the frame, a plurality of traces connect to rails and nubs, which are exposed through the outer mold layer. The rails and nubs act as anodic and cathodic electrodes. When masticated, the non-human animal's mouth (teeth and/or gums) closes the circuit between the anodic and cathodic electrodes, delivering relatively weak direct current electricity to the animal's oral health benefit.

### BACKGROUND

This invention relates generally to the upkeep of oral hygiene in non-human animals. Animals, like humans, experience a variety of oral maladies, including cavity-causing oral microbes, biofilms, gingivitis and periodontitis. It is a goal of the present invention to reduce the occurrences of these maladies, as well as to promote oral health in non-human animals through increasing salivation, promoting gingival tissue regeneration, increasing blood flow in oral tissues, treating systemic oral bacterial diseases, fostering osteogenesis in the bones of the mouth and jaw, and treating other periodontal and oral maladies through non-invasive applications of weak direct current electricity.

Poor oral health, including periodontal diseases, have been shown to correlate with other health problems by doctors and veterinarians. For example, oral health directly has been shown to be an indicator of poor or failing health due to cardiovascular diseases, diabetes, pancreatic diseases, arthritis, and adverse pregnancy outcomes. Thus, methods and devices for improvement of oral health non-human animals are continually sought.

### SUMMARY OF THE INVENTION

The present invention relates to an apparatus for improving the oral health of a non-human animal. More particularly, embodiments of the present invention provide a non-invasive application of relatively weak direct current electricity to the oral cavity of a non-human animal to aid in treating periodontal diseases, killing bacteria, reducing biofilms, increasing salivation, fostering osteogenesis of bones, promoting gingival tissue regeneration, and treating other periodontal and systemic diseases and maladies.

According to an aspect of an embodiment of a device according to the present invention, the device includes an electrical circuit comprising an electrical power source and also includes a frame substantially enclosing the electrical circuit, the frame defining passage therethrough to enable a plurality of electrical couplings to the power source. A first trace is electrically coupled with a first electrical coupling and a second trace is electrically coupled with a second electrically coupled. An electrically insulative body extends longitudinally along an axis from a first end to a second end and surrounds a majority of the enclosure and insulating the first trace from the second trace. The first trace is electrically coupled with a first electrically conductive surface extending through the insulative body and radially outward from the insulative body. The second trace is electrically coupled with a second electrically conductive surface extending through the insulative body and radially outward from the insulative body. The first and/or second trace may be disposed on an outer surface of the frame.

According to another aspect of an embodiment of a device according to the present invention, the electrically conductive first surface comprises a first rail and a first plurality of nubs. Alternatively or additionally, the second electrically conductive surface comprises a second rail and a second plurality of nubs. The first trace may be integrally formed with at least one of the first rail and the first plurality of nubs, and/or the second trace may be integrally formed with at least one of the second and the second plurality of nubs.

According to still another aspect of an embodiment of a device according to the present invention, where first and second pluralities of nubs are provided, the first plurality of nubs and the second plurality of nubs may be positioned in an alignment in a longitudinal direction parallel to the axis. The first plurality of nubs and the second plurality of nubs may alternate (e.g., singularly) along the alignment.

According to yet another aspect of an embodiment of a device according to the present invention, where first and second rails are provided, the first rail and the second rail may extend longitudinally along a majority of a length of the device between the first end and the second end. If a first plurality of nubs and second plurality of nubs are provided in addition to the first rail and the second rail, the first plurality of nubs and the second plurality of nubs may be positioned in an alignment in a longitudinal direction parallel to the axis, the alignment located between the first rail and the second rail.

According to a further aspect of an embodiment of a device according to the present invention, it further includes an enclosure at least substantially containing the power source, the enclosure being received within the frame.

According to still a further aspect of an embodiment of a device according to the present invention, the electrical circuit is defined at least in part by a printed circuit board.

According to an aspect of an embodiment of a method according to the present invention, the method includes the step of providing a device, the device including an electrical circuit comprising an electrical power source and also includes a frame substantially enclosing the electrical circuit, the frame defining passage therethrough to enable a plurality of electrical couplings to the power source. A first trace is electrically coupled with a first electrical coupling and a second trace is electrically coupled with a second electrically coupled. An electrically insulative body extends longitudinally along an axis from a first end to a second end and surrounds a majority of the enclosure and insulating the first trace from the second trace. The first trace is electrically coupled with a first electrically conductive surface extending through the insulative body and radially outward from the insulative body. The second trace is electrically coupled with a second electrically conductive surface extending through the insulative body and radially outward from the insulative body. The first and/or second trace may be disposed on an outer surface of the frame. The method also includes the step of, using the electrical circuit, delivering an electrical current to, through, or across at least one of a tooth, gingival tissue, and saliva located in or about a mouth of the animal.

According to another aspect of an embodiment of a method according to the present invention, the animal is selected from the group consisting of a bovine, ovine, canine, equine, caprine, and porcine.

According to still another aspect of an embodiment of a method according to the present invention, the delivery of electrical current provides at least one of a therapeutic, prophylactic, and regenerative benefit.

### BRIEF DESCRIPTION OF THE DRAWINGS

FIG. 1 is a perspective view of a first embodiment of a device according to the present invention.

FIG. 2 is a front elevation view of the embodiment shown in FIG. 1.

FIG. 3 is a top plan view of the embodiment of FIG. 1.

FIG. 4 is a bottom plan view of the embodiment of FIG. 1.

FIG. 5 is a bottom plan view of the embodiment of FIG. 1, wherein a lid assembly is removed to show a power switch and charging port within.

FIG. 6 is a front elevation view of traces supported on a device frame according to the embodiment shown in FIG. 1.

FIG. 7 is a front elevation view of a device frame according to the embodiment shown in FIG. 1.

FIG. 8 is a front elevation view of an electronics enclosure according to the embodiment shown in FIG. 1.

FIG. 9 is a cross-sectional view of the embodiment of FIG. 1, taken along the 9-9 line of FIG. 2.

### DETAILED DESCRIPTION

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention.

It is known in the art that some oral bacteria cannot survive when exposed to low-microampere direct current electricity. This method of killing oral bacteria and treating bacteria-caused conditions such as gingivitis has been demonstrated in Nachman, U.S. Pat. No. 4,244,373 of Jan. 13, 1981 and in Detsch, U.S. Pat. No. 4,509,519 of Apr. 9, 1985. Killing oral bacteria has the added benefit of preventing tooth decay and dental caries, or cavities. Generally, tooth decay is attributed to aerobic acid-producing bacteria whose acid causes uncompensated demineralization of the teeth. However, Nachman does not instruct optimal approaches to reducing oral bacteria including aerobic and anaerobic bacteria on a species-by-species level and instead teaches a generic, untargeted treatment.

It has been discovered that by delivering a current level preferably in the approximate range of 50 to 500 microamps, with ranges of 100 to 400 microamps, 110-200 microamps, 120-170 microamps, or 140-160 microamps being more preferable, a direct current electrical treatment is able to deliver new and unexpected therapeutic, prophylactic, and regenerative benefits previously unknown in the art. In a specific embodiment, the current is delivered at a generally constant 150 microamps.

Specifically, by utilizing a direct current in the aforementioned range, not only can such a treatment kill bacteria, but it can also kill or disable viruses and fungus as well. Studies from the podiatric field have shown that higher current levels than those used in existing oral electrical treatments are necessary to effectively treat fungal infections (“Low-Voltage Direct Current as a Fungicidal Agent for Treating Onychomycosis”, Kalinowski, et al., Journal of the American Podiatric Medical Association Vol. 94 No. 6:565-572,2004). Thus, fungicidal and viricidal benefits have been additionally provided in conjunction with a method already known to be bactericidal. Studies have shown that these microbicidal properties begin to take effect within approximately 5 and 15 minutes of treatment, reducing both supra- and sub-gingival microbes.

An embodiment 100 of a non-human animal treatment device is shown in the Figures. The treatment device 100 generally includes a body 110 extending longitudinally along a longitudinal axis A, from a first end 110a to a second end 110b. Various structures of the device 100 are generally provided in layers. That is, some structures are manufactured to surround or mate with other structures, providing electrical conductivity from an outer surface of the body to an electrical circuit assembly 170 supported within. The body 110 generally includes a two-shot elastomer including an electrically insulative overmold 120 surrounding an electrically conductive trace layer 130, all supported on a frame 140. Materials used for the overmold 120 and traces 130 preferably have sufficient mechanical resistance to not be chewed through immediately by a non-human animal (e.g., a dog), while also having sufficient elasticity to not be a danger to the non-human animal's dental health, such as through tooth chipping and/or breaking. Materials such as silicone or thermoplastic elastomer (TPE) (preferably having a hardness measurable on the Shore A scale, such as having a Shore A hardness of less than 90) may be suitable to be used in creating the overmold 120 and traces 130, with the traces 130 being at least substantially electrically conductive. The overmold 120 may be manufactured in a variety of shapes and sizes. In the embodiment shown in the Figures, the overmold 120 is preferably manufactured in a bone shape, having a narrow trunk (to mimic an animal bone diaphysis) with flared ends (to mimic animal bone epiphyses with one or more, preferably four, at least substantially symmetrical tubercles). While shown in a decorative shape of an animal bone, it is contemplated to provide a device 100 in other shapes, such as a ball, a cylinder, or a half-bone, which may further include a handle for human manual manipulation during animal mastication.

Extending into the body 110 (and preferably into the frame 140) from the first end 110a is preferably a first cavity, or reentrant bore, 124. In use, the cavity 124 may receive digestible contents for a non-human animal, such as food (e.g., kibble, peanut butter, etc.) and/or medicine. Such digestible contents may encourage the non-human animal to interact with the device 100 and specifically to chew on the device 100 for purposes to be described below. The cavity 124 may optionally include flaps 126 extending radially inward to at least partially cover the cavity 124, which allow digestible contents to be placed into the cavity 124 with ease while resisting the removal of such digestible contents from the cavity 124, further encouraging mastication of the device 100.

Located at the second end 110b of the body 110 is preferably a lid assembly 122 cooperating with the frame 140 to cover a second cavity 146 in the frame 140. Such lid assembly 122 is preferably manually removable from the frame 140 to provide access into the body 110. In the embodiment shown in the Figures, both the frame 140 and the lid assembly 122 comprise mating threads, which allows the lid assembly 122 to screw into the frame 140 and be held in place thereby. The lid assembly 122 preferably protects electrical circuitry and/or an electrical circuit enclosure, to be described later, from wear and other damage that may occur to the body 110 of the device 100.

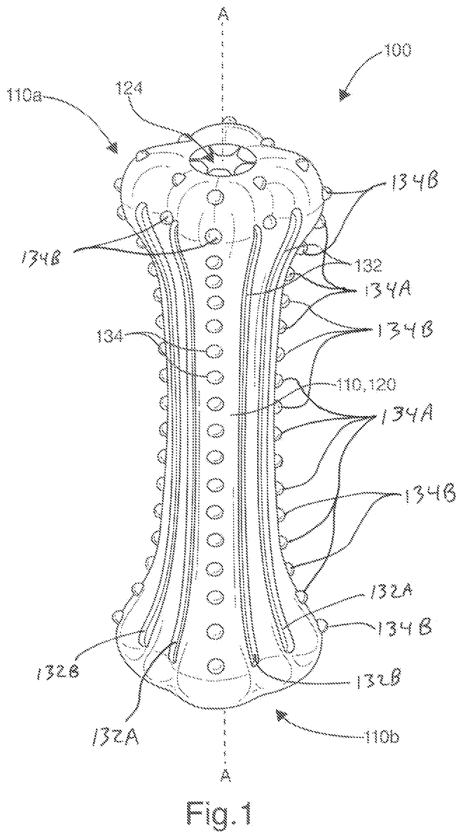

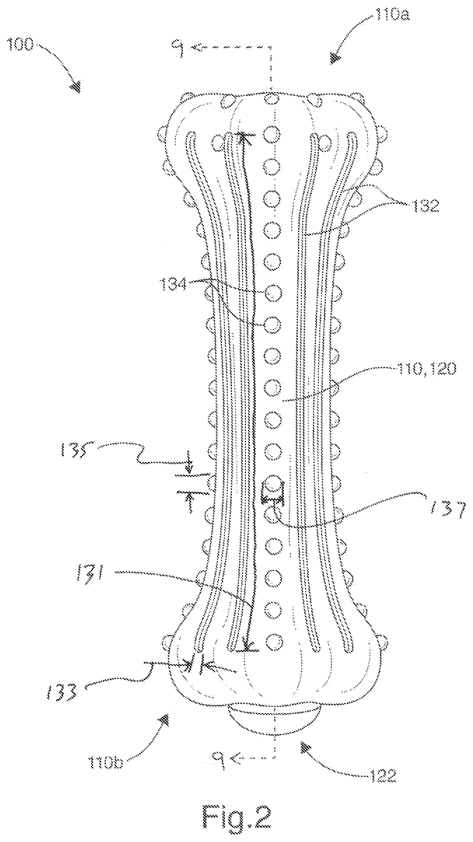

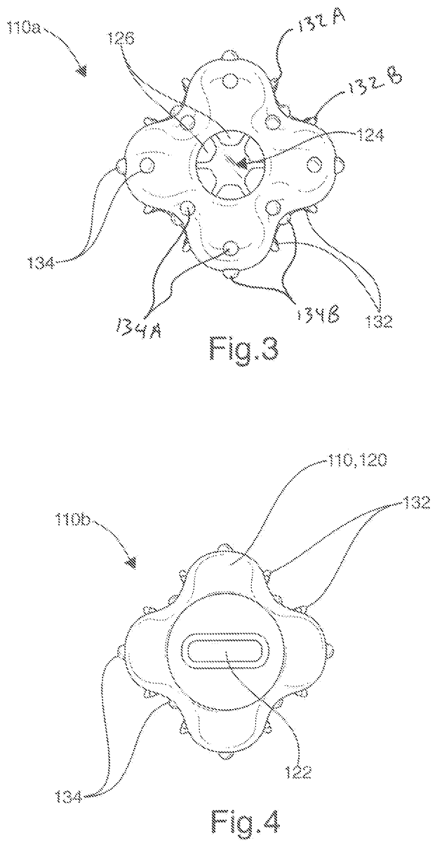

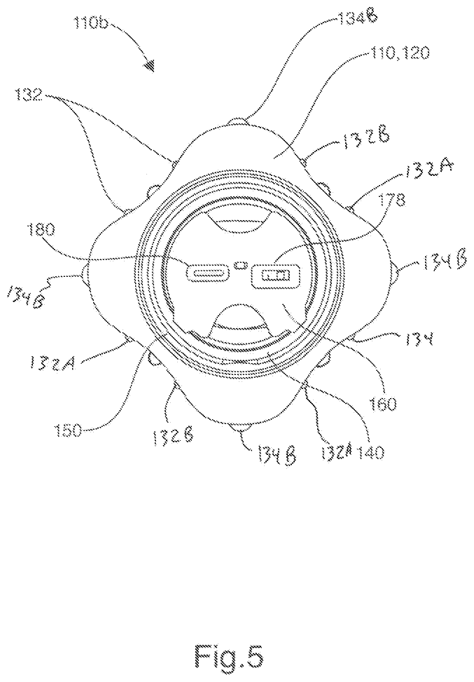

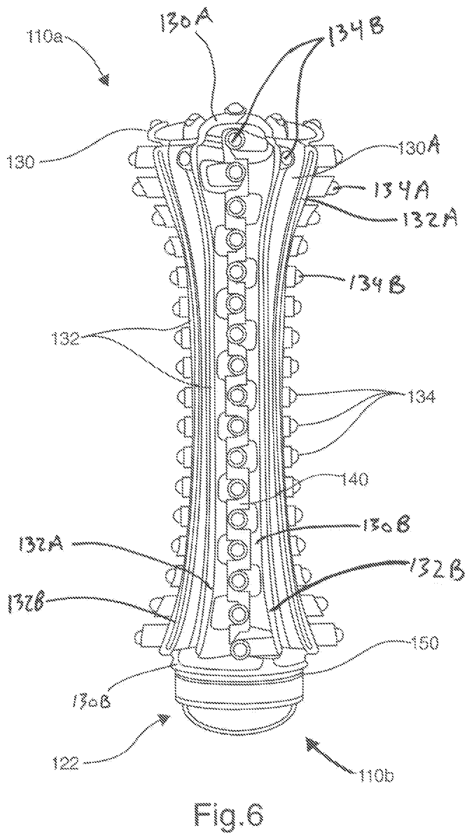

Preferably protruding through the overmold 120 are a plurality of electrically conductive surfaces, which may be formed in rails 132 and/or nubs 134 electrically coupled to, and preferably formed integrally with, the traces 130 that run beneath the overmold 120, the overmold insulating the rails 132A and/or nubs 134A of a first polarity 132A from the rails 132B and/or nubs 134B of a second polarity. As used herein, the term “rail” refers to a structure that has a rail length 131 (measured along an external surface thereof) that is substantially larger than a rail width 133 (measured along the external surface and perpendicular to the length), such as a rail length 131 equal to five or more times the rail width 133. As used herein, the term “nub” refers to a structure that has a nub length 135 (measured along an external surface thereof) that is substantially similar to a nub width 137 (measured along the external surface and perpendicular to the length), such as a nub length 135 equal to less than five times the nub width 137. Each rail of the plurality of rails 132 is preferably substantially similar to the other rail(s) 132 in length, width, and height (radial extension outward from axis A beyond the overmold 120), each running longitudinally along the body 110 of the device 100 generally in a direction parallel to the longitudinal axis A and to each other. The plurality of rails 132 are preferably provided in pairs, as seen in FIG. 1. In such a pair, during operation, one rail 132A is preferably oppositely electrically charged to the other rail 132B. In other words, one rail 132A in each pair is preferably coupled to one pole (positive or negative) of the power source 174, while the other rail 132B is preferably coupled to the other pole (negative or positive) of the power source 174. When a non-human animal bites or licks the device 100, the construction allows a circuit to be closed, as between the anodic portions, cathodic portions, oral tissue, and/or saliva, delivering an electric current through to the mouth of the animal or along oral tissue of the animal.

Like the rails 132, during use, the nubs 134 are also preferably electrically coupled to one pole or the other of the power source. Each nub 134 is also preferably substantially similar to each other nub 134 in form factor. The nubs 134 are preferably arranged in a single line between two pairs of rails 132A, B, extending onto the first end of the body 110, as seen in FIG. 1. In such arrangement, the nubs 134 preferably alternate in electric charge along the line, such that two nubs 134A, 134B in close proximity to each other are always oppositely charged. The alternating pattern of the nubs 134A, B works for the same reasons as the pair of rails 132A, B, increasing the chances that the non-human animal will complete the circuit between two proximate nubs 134A, 134B of opposite charges to deliver an electrical current through to the mouth of the animal or along oral tissue of the animal. While singular nub alternation is preferred, multiple alternation (two or more of one nub polarity 134A may be provided in alternating alignment with one or more of the other nub polarity 134B or vice versa).

Other embodiments of the present invention may feature alternate arrangements to the one shown in the Figures and described herein. For example, alternate embodiments of the present invention may feature rows of nubs 134 of the same polarity and/or pairs of rails 132 wherein each rail 132 has the same polarity.

As mentioned before, the plurality of nubs 134 and rails 132 are electrically coupled to traces 130, which run beneath the mold 120. One set of traces 130 electrically connects each anodic nub 134 and rail 132, while a second, separate set of traces 130 electrically connects each cathodic nub 134 and rail 132, giving each single nub 134 or rail 132 of the plurality of nubs 134 and rails 132 their respective electrical charge. Each set of traces 130 is preferably electrically and mechanically continuous, such that one trace 130 connects all anodic nubs 134 and rails 132, while the other trace 130 connects all cathodic nubs 134 and rails 132. Alternatively, other embodiments of the present invention may feature multiple traces 130 that are in electronic communication with the anodic nubs 134 and rails 132 and/or multiple traces 130 in electronic communication with the cathodic nubs 134 and rails 132.

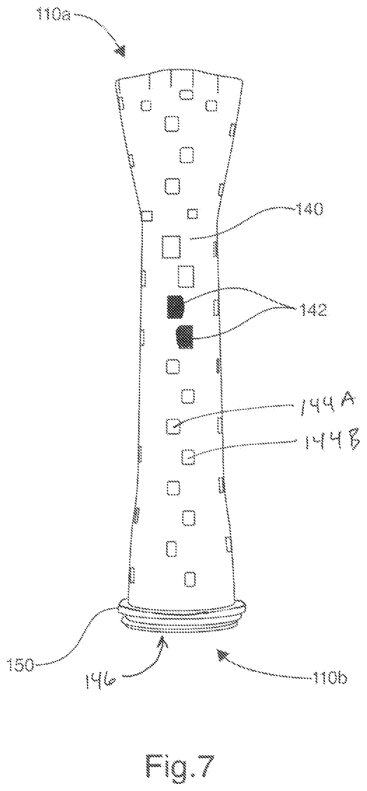

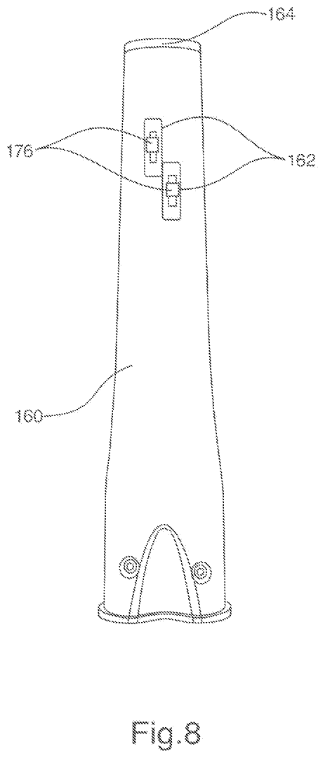

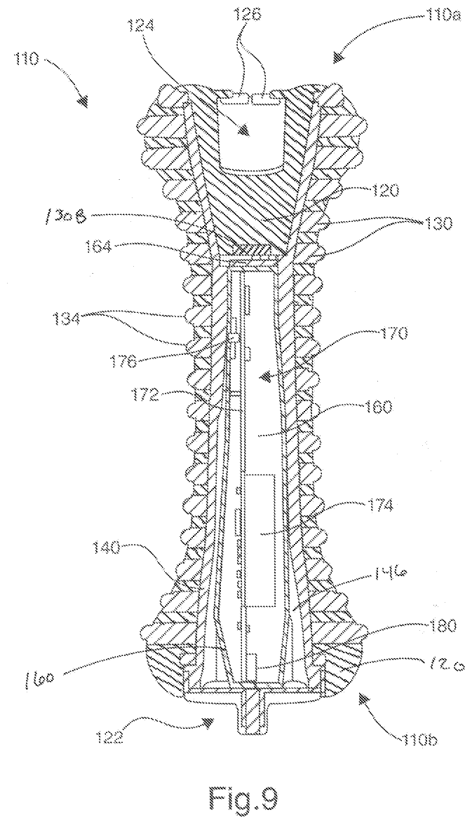

Preferably inside a majority of the traces 130 is the frame 140, which can be seen with particular reference to FIGS. 7 and 9. The frame 140 is preferably manufactured (e.g., machined, printed or molded) plastic to protect the innermost layers of the device 100 from being damaged from the non-human animal chewing on the device 100. The frame 140 is preferably a single piece of plastic and may be created by processes such as injection molding or machining or 3-dimensional printing. One end of the frame 140, preferably near the distal end 110b of the device 100, is preferably open, such that the electrical circuit assembly 170 and other contents within the frame 140 may be removed without having to break or otherwise malform the frame 140. The frame 140 also preferably comprises at least one (possibly more than one) hole 142 through which the traces 130 may electrically and/or mechanically connect to the electrical circuit assembly 170 located within the frame 140. Finally, the distal end 110b of the frame 140 may be threaded in the same manner as the mold 120 to allow the lid assembly 122 to removably couple with the frame 140 for extra support. A plurality of other indentations 144A, B (or at least partial mechanical interlocks) are provided in an outer surface of the frame 140 to receive a portion of a first shot elastomer during manufacture, further described below, so as to aid in preventing delamination. A gasket 150 may also optionally be provided proximate the threaded portion of the frame 140 to seal the connection between the lid assembly 122 and the frame 140.

Within the frame 140, the device 100 may include an enclosure 160, which renders further enclosed electronic circuit assembly 170 substantially modular. The enclosure 160 is preferably hard plastic and may comprise two separate pieces that are preferably removably attached to each other using fasteners (e.g. nuts and bolts) or integrally molded mating clip/slot formations (not shown). For example, the enclosure 160 may comprise a top piece and bottom piece, or left piece and right piece, that may be removably secured to allow access to the electronic circuit assembly 170 within. The enclosure 160 also preferably comprises channels 162, which permit electronic trace contacts 176 to connect the electrical circuit assembly 170 within to the traces 130 outside of the enclosure 160, the traces 130 extending through the openings 142. Finally, the enclosure 160 may optionally further comprise other safety technology, such as a foam compression pad 164, to further protect the enclosed electrical circuit assembly 170 for damage.

Disposed within the frame 140 and/or the enclosure 160 is the electrical circuit assembly 170, which comprises a printed circuit board (PCB) 172, power supply 174, electronic trace contacts 176, and a power switch 178. The power supply 174 may be a simple dry-cell battery, a rechargeable battery, a capacitor, a kinetic energy generator, a piezoelectric generator, a microcontrolled DC power supply, or other power supply (such as a microcontrolled AC power supply). Regardless of the type of power supply 174 used, it is most preferable to control the amount of electrical current delivered by the treatment device to provide a relatively constant current power source to provide at least up to about 200 microamps, and preferably up to 500 microamps, of direct or alternating current. While 50 microamps to about 500 microamps may be a desired range, about 50 microamps to about 250 microamps is preferred, and about 150 microamps is still further preferred. In embodiments comprising a rechargeable battery power source 174, the PCB 172 may further comprise a charging port 180, such as a USB-A, USB-B, or USB-C charging port.

Fully contemplated within the purview of the present invention to be included in the circuit assembly 170 are timers, audible (e.g., sounds representative or imitative of a mouse squeak or bird chirp), tactile/haptic (e.g. vibrations provided by a vibratory motor such as a coin or pancake vibration motor), and/or visible prompts or feedback, or usage, activity and/or power indicators (e.g. beeper, buzzer, light-emitting diodes), motion activation (e.g. using an accelerometer), moisture activation, pressure activation, electrical current intensity adjustment (e.g., based on sensed impedance between a cathode and an anode), and/or the power switch 178 to control the possible current delivery by the power supply 174.

The power switch 178 may be a binary power switch or push-button switch known in the art. In manufacturing, the power switch 178 and charging port 180 are preferably affixed to the PCB 172 proximate the distal end 110b of the device body 110, such that removal of the lid assembly 122 allows access to the power switch 178 and/or charging port 180 without having to take the device 100 apart or otherwise remove any other parts of the device 100.

The PCB 172 draws power from the power supply 174 through its various components and electrical connections before outputting electrical charges through the electronic trace contacts 176. One of the electronic trace contacts 176 is positively charged and one is negatively charged. These contacts 176 are aligned with the channels 162 in the enclosure 160 and hole(s) 142 in the frame 140, allowing electronic and mechanical communication with the traces 130. Thus, one set of traces 130, and the nubs 134 and rails 132 electronically and mechanically connected to it, become anodes, while the other set and its associated nubs 134 and rails 132 become cathodes.

The coupling of the anodic and cathodic traces 130 to the power supply 174 is not required to be a direct coupling, but rather may be indirect coupling through a variety of other electrical passive or active electrical components, such as one or more voltage regulators, operational amplifiers, transistors, microcontrollers, voltage converters/inverters, etc. Regardless of the specific circuit design, it is preferable that the circuit assembly 170 be able to supply a stimulation (pulsed or steady) current of about 50 microamps to about 500 microamps to a load (Ro) of up to about 70 kilo-ohms. More preferably, to such load, a stimulation current of about 50-250 microamps, and still more preferably, a current of about 150 microamps has been shown to be effective. A circuit will be completed by an animal's mouth when the device 100 is masticated.

In use, the lid assembly 122 may be removed to access the charging port 180 and power switch 178. When the device 100 is charged, the power switch 178 may be activated to turn on the device 100. The device 100 is then preferably given to a non-human animal to play with/masticate. When the animal bites down on the device 100, there its teeth and/or gums and/or saliva will contact both an anode and a cathode at once, completing the circuit and delivering the stimulant current, preferably between 50 and 250 microamps and most preferably approximately 150 microamps, to the animal's mouth. As discussed above, this electrical charge has numerous health benefits, including therapeutic, prophylactic, and regenerative benefits previously unknown in the art. It is further contemplated by the present invention that the treatment apparatus 100 is preferably used by non-human animals, such as felines, bovines, ovines, canines, equines, porcines, etc.

To make the device 100, the frame 140 is molded, machined or otherwise formed, preferably of plastic, and in a general form capable of supporting elastomer overmolding. In a preferred shape, the frame 140 is generally provided in a back-to-back cupule form sharing an intermediate terminus separating two cavities. The frame 140 is placed in a first mold providing a void to be filled with an electrically conductive elastomer to form the traces 130 and the conductive surface(s) (e.g., nubs 132 and rails 134). The conductive elastomer is injected into the first mold and the conductive elastomer is allowed to sufficiently cure. A second mold is used to provide a void to be filled with an electrically insulative elastomer to form the insulative body 110. The insulative elastomer is injected into the second mold (which contains the frame 140 and traces 130) and the insulative elastomer is allowed to sufficiently cure. The electronic circuit (including power supply) is placed within the frame such that the traces 130 are electrically coupled with the circuit through the frame 140 and through the enclosure 160 (if provided)

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

We claim:

1. A device comprising:
an electrical circuit comprising an electrical power source;
a frame substantially enclosing the electrical circuit, the frame defining passage therethrough to enable a plurality electrical couplings to the power source;
a first trace electrically coupled with a first of the electrical couplings;
a second trace electrically coupled with a second of the electrical couplings; and
an electrically insulative body extending longitudinally along an axis from a first end to a second end and surrounding a majority of the enclosure and insulating the first trace from the second trace,
wherein the first trace is electrically coupled with a first electrically conductive surface extending through the insulative body and radially outward from the insulative body, and
wherein the second trace is electrically coupled with a second electrically conductive surface extending through the insulative body and radially outward from the insulative body.

2. The device according to claim 1, wherein the first electrically conductive surface comprises a first rail and a first plurality of nubs.

3. The device according to claim 1, wherein the second electrically conductive surface comprises a second rail and a second plurality of nubs.

4. The device according to claim 3, wherein the first electrically conductive surface comprises a first rail and a first plurality of nubs.

5. The device according to claim 4, wherein the first plurality of nubs and the second plurality of nubs are positioned in an alignment in a longitudinal direction parallel to the axis.

6. The device according to claim 5, wherein the first plurality of nubs and the second plurality of nubs alternate along the alignment.

7. The device according to claim 6, wherein the first plurality of nubs and the second plurality of nubs singularly alternate along the alignment.

8. The device according to claim 4, wherein the first rail and the second rail extend longitudinally along a majority of a length of the device between the first end and the second end.

9. The device according to claim 8, wherein the first plurality of nubs and the second plurality of nubs are positioned in an alignment in a longitudinal direction parallel to the axis, the alignment located between the first rail and the second rail.

10. The device according to claim 4, wherein the first trace is integrally formed with at least one of the first rail and the first plurality of nubs.

11. The device according to claim 4, wherein the second trace is integrally formed with at least one of the second rail and the second plurality of nubs.

12. The device according to claim 11, wherein the first trace is integrally formed with at least one of the first rail and the first plurality of nubs.

13. The device according to claim 1, further comprising an enclosure at least substantially containing the power source, the enclosure being received within the frame.

14. The device according to claim 1, wherein the electrical circuit is defined at least in part by a printed circuit board.

15. The device according to claim 1, wherein a majority of the first trace is disposed on an outer surface of the frame.

16. The device according to claim 1, wherein a majority of the second trace is disposed on an outer surface of the frame.

17. The device according to claim 16, wherein a majority of the first trace is disposed on the outer surface of the frame.

18. A method comprising the steps of:
providing a device according to claim 1 to a domesticated animal; and
using the electrical circuit, delivering an electrical current to, through, or across at least one of a tooth, gingival tissue, and saliva located in or about a mouth of the animal.

19. A method according to claim 18, wherein the animal is selected from the group consisting of a bovine, ovine, canine, equine, caprine, and porcine.

20. A method according to claim 18, wherein the delivery of electrical current provides at least one of a therapeutic, prophylactic, and regenerative benefit.
